SEQUENCE LISTING (1 sequence)

SEQ ID NO: 1 - protein, 366 aa, Homo sapiens
MMGSVLPAEALVLKTGLKAPGLALAEVITSDILHSFLYGRWRNVLGEQLFEDKSHHASPKTAFTAEVLAQSFSGEVQKLSSLVLPAEVIIAQSSIPGEGLGIFSKTWIKAGTEMGPFTGRVIAPEHVDICKNNNLMWEVFNEDGTVRYFIDASQEDHRSWMTYIKCARNEQEQNLEVVQIGTSIFYKAIEMIPPDQELLVWYGNSHNTFLGIPGVPGLEEDQKKNKHEDFHPADSAAGPAGRMRCVICHRGFNSRSNLRSHMRIHTLDKPFVCRFCNRRFSQSSTLRNHVRLHTGERPYKCQVCQSAYSQLAGLRAHQKSARHRPPSTALQAHSPALPAPHAHAPALAAAAAAAAAAAHHLPAMVL

(19) United States
(12) Patent Application Publication
(10) Pub. No.: US 2024/0302351 A1
(43) Pub. Date: Sep. 12, 2024

(54) PLATFORM FOR TARGET DISCOVERY AND METHODS OF USE THEREOF

(71) Applicant: Doloromics Inc, Menlo Park, CA (US)

(72) Inventors: Candler Paige, Foster City, CA (US); Christopher Flores, San Diego, CA (US); Jackson Brougher, Belmont, CA (US); Dale George, Menlo Park, CA (US); Theodore Price, Dallas, TX (US)

(21) Appl. No.: 18/589,056

(22) Filed: Feb. 27, 2024

Related U.S. Application Data
(60) Provisional application No. 63/448,779, filed on Feb. 28, 2023.

(51) Int. Cl. G01N 33/50; C12N 5/074; C12N 5/0793; G01N 33/487

(52) U.S. Cl. CPC G01N 33/5058; C12N 5/062; C12N 5/0696; G01N 33/48728; G01N 33/5023; G01N 33/5073; G01N 2333/4703; G01N 2500/10

(57) ABSTRACT

Provided herein is a recombinant cell for drug candidate screening. Also provided herein is a method of producing the recombinant cell, and a method of identifying a drug candidate comprising providing the recombinant cell.

### CROSS-REFERENCE

This application claims priority to U.S. Provisional Application No. 63/448,779, filed Feb. 28, 2023, the contents of which is incorporated herein in its entirety.

### SEQUENCE LISTING

The instant application contains a Sequence Listing which has been submitted electronically in XML file format and is hereby incorporated by reference in its entirety. Said XML copy, crated on Apr. 22, 2024, is named 60329-706_201_SL.xml and is 2,262 bytes in size.

### BACKGROUND OF THE INVENTION

The neurological and medical determinants of an individual's pain sensitivity and perception of pain are influenced by a wide variety of factors comprising genetic, epigenetic, transcriptomic, and interactomic (e.g., related to the interactions between ligands and receptors within a cell) factors. Many current treatments for pain are addictive, lose efficacy over time, and do not treat the source of the pain sensitivity or perception. Described herein is a recombinant cell that is capable of proliferation in vitro and that replicates features of cells that are associated with the sensation and perception of pain in vivo. Also described herein is a method of identifying a drug candidate for treating pain comprising contacting the recombinant cell with a drug candidate.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### SUMMARY OF THE INVENTION

In some aspects, provided herein is a recombinant cell comprising an exogenous gene, the exogenous gene encoding a polypeptide comprising an amino acid sequence having at least 60% sequence identity to an amino acid sequence of SEQ ID NO. 1 or an active fragment thereof, wherein the recombinant cell is capable of proliferating in vitro.

In some aspects, provided herein is a recombinant cell comprising an exogenous gene, the exogenous gene encoding a positive regulatory (PR) domain zinc finger protein 12 (PRDM12) polypeptide, wherein the recombinant cell is capable of proliferating in vitro.

In some embodiments, the recombinant cell forms colonies that propagate repeatedly.

In some embodiments, the recombinant cell is a stem cell.

In some embodiments, the recombinant cell is a pluripotent stem cell (PSC).

In some embodiments, the recombinant cell is an induced pluripotent stem cell (iPSC). In some embodiments, the recombinant cell is a human induced pluripotent stem cell (hiPSC).

In some embodiments, the recombinant cell is a neural cell. In some embodiments, the neural cell is a neural progenitor cell (NPC). In some embodiments, the neural cell is a neuron. In some embodiments, the neuron is a sensory neuron. In some embodiments, the neuron is an afferent sensory neuron. In some embodiments, the neuron is a nociceptive lineage sensory neuron. In some embodiments, the neuron is a nociceptive sensory neuron. In some embodiments, the neuron is a nociceptor-like sensory neuron. In some embodiments, the neuron is a nociceptor.

In some embodiments, the polypeptide comprises an amino acid sequence with at least 80% sequence identity to SEQ ID NO: 1. In some embodiments, the polypeptide comprises an amino acid sequence with at least 85% sequence identity to SEQ ID NO: 1. In some embodiments, the polypeptide comprises an amino acid sequence with at least 90% sequence identity to SEQ ID NO: 1. In some embodiments, the polypeptide comprises an amino acid sequence with at least 95% sequence identity to SEQ ID NO: 1. In some embodiments, the polypeptide comprises an amino acid sequence with at least 100% sequence identity to SEQ ID NO: 1.

In some embodiments, the polypeptide has DNA binding activity.

In some embodiments, the polypeptide comprises a transcription factor domain.

In some embodiments, the polypeptide has methyltransferase activity.

In some embodiments, the polypeptide comprises a zinc finger domain.

In some embodiments, the polypeptide comprises a PR domain.

In some aspects, provided herein is a method of identifying a drug candidate for treating pain (e.g., in a pain-related disorder), the method comprising a) providing a recombinant cell comprising an exogenous gene, the exogenous gene encoding a polypeptide comprising an amino acid sequence having at least 60% sequence identity to an amino acid sequence of SEQ ID NO. 1 or an active fragment thereof, wherein the recombinant cell is capable of proliferating in vitro; b) culturing the recombinant cell in vitro; c) contacting the recombinant cell with the drug candidate; d) evaluating at least one of (i) an electrophysiological characteristic of the recombinant cell, (ii) a proteomic characteristic of the recombinant cell, (iii) a transcriptomic characteristic of the recombinant cell, (iv) a metabolomic characteristic of the recombinant cell, (v) a cell morphology characteristic of the recombinant cell, (vi) an axonal outgrowth characteristic of the recombinant cell, or (vii) cell viability, thereby providing a first measurement; and e) determining a drug candidate score for the drug candidate based at least in part on the first measurement.

In some aspects, provided herein is a method of identifying a drug candidate for treating pain (e.g., in a pain-related disorder), the method comprising: a) providing a recombinant cell comprising an exogenous gene, the exogenous gene encoding a PR domain zinc finger protein 12 (PRDM12) polypeptide, wherein the recombinant cell is capable of proliferating in vitro; b) culturing the recombinant cell in vitro; c) contacting the recombinant cell with the drug candidate; evaluating at least one of (i) an electrophysiological characteristic of the recombinant cell, (ii) a proteomic characteristic of the recombinant cell, (iii) a transcriptomic characteristic of the recombinant cell, (iv) a metabolomic characteristic of the recombinant cell, (v) a cell morphology characteristic of the recombinant cell, (vi) an axonal outgrowth characteristic of the recombinant cell, or (vii) cell viability, thereby providing a first measurement; determining a drug candidate score for the drug candidate based at least in part on the first measurement.

In some embodiments, the characteristic is evaluated when the recombinant cell is in contact with the drug candidate or after incubating the recombinant cell with the drug candidate.

In some embodiments, the determining of the drug candidate score comprises (at least in part) evaluating the first measurement and a second measurement. In some embodiments, the second measurement is a standard score for the recombinant cell (e.g., obtained from a database). In some embodiments, the second measurement is obtained (at least in part) by evaluating the characteristic before contacting the recombinant cell with the drug candidate.

In some embodiments, in some embodiments, the recombinant cell is any one of the recombinant cells provided herein.

In some embodiments, the first electrophysiological characteristic is selected from the group consisting of: current through ion channels (e.g., while holding the membrane voltage at a defined level), ion-channel conductance (e.g., while holding the membrane voltage at a defined level), membrane conductance (e.g., while holding the membrane voltage at a defined level), change in membrane conductance (e.g., while holding the membrane voltage at a defined level), membrane potential, change in membrane potential, rate of action potentials, change in rate of action potentials, timing of action potentials, change in timing of action potentials (e.g., a change in action potential firing rate), rate of postsynaptic potentials, change in rate of postsynaptic potentials, timing of postsynaptic potentials, and change in timing of postsynaptic potentials.

In some embodiments, the first proteomic characteristic is selected from the group consisting of: protein expression, change in protein expression, protein expression levels, change in protein expression levels, cell membrane expression of proteins, posttranslational modification of proteins, change in posttranslational modification of proteins, ligand-receptor interactions, change in ligand-receptor interactions.

In some aspects, provided herein is a method of recombinantly producing a genetically modified cell, the method comprising: a) providing an isolated cell; and b) modifying the isolated cell with one or more exogenous genes, wherein the modified cell comprises one or more phenotypic characteristics specific to a sensory neuron.

In some embodiments, the modification is performed via a gene knock-in.

In some embodiments, the modification is a knock-in of a PRDM gene. In some embodiments, the PRDM gene is PRDM12. In some embodiments, the PRDM knock-in is at the AAVS1 locus.

In some embodiments, the gene editing is performed using an engineered nuclease. In some embodiments, the engineered nuclease is selected from the group consisting of: (i) a zinc finger nuclease (ZFN), (ii) a transcription activator-like effector nuclease (TALEN), and (iii) an RNA-guided engineered nuclease (RGEN) that includes a Cas protein and a guide RNA that specifically binds to a particular sequence of the PRDM genes.

In some embodiments, the gene editing is performed by introducing into the cell a nucleic acid encoding a Cas protein or the Cas protein itself, and either (i) a guide RNA specifically binding to a particular sequence of the PRDM gene or (ii) DNA encoding the guide RNA that specifically binds to a particular sequence of the PRDM gene. In some embodiments, the guide RNA is a dual RNA comprising crRNA and tracrRNA, or a single-strand guide RNA.

In some embodiments, the gene editing is performed using a CRISPR/Cas system. In some embodiments, the CRISPR/Cas system is type I. In some embodiments, the CRISPR/Cas system comprises Cas3. In some embodiments, the CRISPR/Cas system is type II. In some embodiments, the CRISPR/Cas system comprises Cas9. In some embodiments, the CRISPR/Cas system is type V. In some embodiments, the CRISPR/Cas system comprises Cpf1.

In some embodiments, the cell is a stem cell. In some embodiments, the stem cell is an induced pluripotent stem cell (iPSC), embryonic stem cell (ES), somatic cell nuclear transfer derived embryonic stem cell, or adult stem cell. In some embodiments, the stem cell is an induced pluripotent stem cell (iPSC). In some embodiments, the iPSC is an XCL-1 iPSC.

In some embodiments, the method further comprises comprising differentiating the cell into a nociceptor-like neuron.

In some aspects, provided herein is an isolated neuronal cell population derived from differentiating a genetically modified cell produced according to any one of the methods described herein.

In some aspects, provided herein is an isolated neuronal cell population comprising the nociceptor-like neuron produced according to any one of the methods described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

The identification of potential drug candidates in the treatment pain is hindered by the inaccessibility of neurons that transduce, encode, or convey noxious-stimuli or painful-stimuli. The identification of drug candidates is further limited by the use of primary cells from postmortem neural samples. Primary cells from postmortem neural samples suffer from significant shortcomings such as short lifespan in vitro and non-proliferation. A platform that replicates the fundamental features of pain-related neural cells is needed. The positive regulatory (PR) domain zinc finger protein 12 (PRDM12) polypeptide influences the development of nerve cells that assist in perception and sensation of pain (alternatively named nociceptive nerve cells or nociceptors), and PRDM12 is expressed in adult human nociceptors. Provided herein is a recombinant cell that is capable of proliferating in vitro, and that expresses an exogenous polypeptide comprising an amino acid sequence having at least 60% sequence identity to an amino acid sequence of SEQ ID NO. 1 or an active fragment thereof, or that expresses an exogenous PRDM12 polypeptide or an active fragment thereof. Also provided herein is a method of identifying a drug candidate for treating pain, the method comprising providing a recombinant cell that is capable of proliferating in vitro, and that expresses an exogenous polypeptide comprising an amino acid sequence having at least 60% sequence identity to an amino acid sequence of SEQ ID NO. 1 or an active fragment thereof, or expresses an exogenous PRDM12 polypeptide or an active fragment thereof. In some instances, SEQ ID NO. 1 is in Table 1.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

Any ranges listed herein are intended to be inclusive of endpoints. For example, a range of 2 to 4 includes 2 and 4.

As used herein, the singular forms “a,” “an,” and “the” include plural references unless the content clearly dictates otherwise. For example, the term “a sample” includes a plurality of samples, including mixtures thereof.

The terms “about” or “approximately,” when immediately preceding a numerical value, refer to ±10% of the value provided. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30%”) refers, respectively to all values in the series, or the end points of the range.

As used herein, the terms “including,” “includes,” “having,” “has,” “with,” or variants thereof are intended to be inclusive in a manner like the term “comprising.”

In some embodiments, provided herein is a recombinant cell comprising an exogenous gene. In some embodiments, the exogenous gene encodes a polypeptide comprising an amino acid sequence having at least 60% sequence identity to an amino acid sequence of SEQ ID NO. 1. In some embodiments, the encoded exogenous polypeptide may have at least about 65% sequence identity to SEQ ID NO. 1. In some embodiments, the encoded exogenous polypeptide may have at least about 70% sequence identity to SEQ ID NO. 1. In some embodiments, the encoded exogenous polypeptide may have at least about 75% sequence identity to SEQ ID NO. 1. In some embodiments, the encoded exogenous polypeptide may have at least about 80% sequence identity to SEQ ID NO. 1. In some embodiments, the encoded exogenous polypeptide may have at least about 85% sequence identity to SEQ ID NO. 1. In some embodiments, the encoded exogenous polypeptide may have at least about 90% sequence identity to SEQ ID NO. 1. In some embodiments, the encoded exogenous polypeptide may have at least about 95% sequence identity to SEQ ID NO. 1. In some embodiments, the encoded exogenous polypeptide may have at least about 100% sequence identity to SEQ ID NO. 1.

The term “percent (%) sequence identity,” with respect to a reference polypeptide sequence, is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known, for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. The % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in this paragraph using the ALIGN-2 computer program.

In some embodiments, the recombinant cell provided herein comprises a polypeptide comprising an amino acid sequence having at least 60% sequence identity to an amino acid sequence of SEQ ID NO. 1 and having a length of at least about 150 amino acid residues.

In some embodiments, the recombinant cell provided herein comprises a polypeptide comprising an amino acid sequence having at least 60% sequence identity to an amino acid sequence of SEQ ID NO. 1, the expression of the polypeptide under the control of the elongation factor 1-alpha (EF1-α) promoter.

In some embodiments, the recombinant cell provided herein comprises an exogenous gene, the exogenous gene encoding a polypeptide that comprises a methyltransferase domain. In specific embodiments, the methyltransferase domain comprises a Su(var)3-9, Enhancer-of-zeste and Trithorax (SET) methyltransferase domain. In some embodiments, the SET domain comprises a positive regulatory domain I-binding factor 1 (PRDI-BF1) and a retinoblastoma protein-interacting zinc finger gene 1 (RIZ1) homologous containing (PRDF1-RIZ or PR) domain.

In some embodiments, the recombinant cell provided herein comprises an exogenous gene, the exogenous gene encoding a polypeptide that comprises a positive regulatory (PR) domain. In some embodiments, the PR domain is related to a SET methyltransferase domain. In some embodiments, the PR domain is a subtype of the SET methyltransferase domain. In some embodiments, the PR domain is a variant of a SET methyltransferase domain. In some embodiments, the PR domain is a SET methyltransferase domain.

In some embodiments, the cell provided herein comprises an exogenous gene, the exogenous gene encoding a polypeptide that comprises a zinc finger domain. In some embodiments, the cell provided herein comprises an exogenous gene, the exogenous gene encoding a polypeptide that comprises two zinc finger domains. In some embodiments, the cell provided herein comprises an exogenous gene, the exogenous gene encoding a polypeptide that comprises three zinc finger domains. In some embodiments, the cell provided herein comprises an exogenous gene, the exogenous gene encoding a polypeptide that comprises at least three zinc finger domains. In some embodiments, the one or more zinc finger domains are C2H2-type zinc finger domains.

In some embodiments, the cell provided herein comprises an exogenous gene, the exogenous gene encoding a polypeptide that comprises a polyalanine tract. In some embodiments, the cell provided herein comprises an exogenous gene, the exogenous gene encoding a polypeptide that comprises a C-terminal polyalanine tract. In some embodiments, the C-terminal polyalanine tract comprises six (6) to fourteen (14) alanine residues.

In some embodiments, the cell provided herein comprises an exogenous gene, the exogenous gene comprising an expansion of tri-nucleotide alanine codons. In some embodiments, the cell provided herein comprises an exogenous gene, the exogenous gene encoding a polypeptide that comprises a polyalanine expansion. In specific embodiments, the cell provided herein comprises an exogenous gene, the exogenous gene encoding a polypeptide that comprises a C-terminal polyalanine tract expansion. In some embodiments, the C-terminal polyalanine tract comprises at least 15 alanine residues.

In some embodiments, the recombinant cell provided herein comprises an exogenous gene, the exogenous gene encoding a positive regulatory (PR) domain zinc finger protein 12 (PRDM12) polypeptide. The term “PRDM12 polypeptide” includes the polypeptide encoded by the PRDM12 gene (also known as the HSAN8 or the PFM9 gene), the PRDM12 gene comprising mutations, variants of the PRDM12 gene, and alleles of the PRDM12 gene. The PRDM12 polypeptide may also be referred to as PRDI-BF1 and RIZ homology domain-containing protein 12. In some embodiments, the PRDM12 polypeptide has a length of at least about 150 amino acid residues.

In some embodiments, the recombinant cell provided herein comprises an exogenous gene, the exogenous gene encoding a positive regulatory (PR) domain zinc finger protein 12 (PRDM12) polypeptide, the expression of the polypeptide under the control of the elongation factor 1-alpha (EF1-α) promoter.

In some embodiments, the PRDM12 polypeptide described herein has DNA binding activity. In some embodiments, the PRDM12 polypeptide has methytransferase activity. In some embodiments, the PRDM12 polypeptide has histone methytransferase activity. In some embodiments, the PRDM12 polypeptide has metal ion binding activity. In some embodiments, the PRDM12 polypeptide has zinc ion (Zn²⁺) binding activity.

In some embodiments, the PRDM12 polypeptide has the activity of a zinc-finger. In some embodiments, the PRDM12 polypeptide has the activity of a Kruppel-like zinc finger protein. In some embodiments, the PRDM12 polypeptide has the activity of a C2H2-type zinc finger protein.

In some embodiments, the PRDM12 polypeptide described herein has transferase activity. In some embodiments, the PRDM12 polypeptide has methyltransferase activity. In some embodiments, the PRDM12 polypeptide has histone methyltransferase activity. In specific embodiments, the PRDM12 polypeptide has histone H3-K9 methyltransferase activity. In some embodiments, the PRDM12 polypeptide has the activity of positive regulation of H3-K9 methylation. In some embodiments, the PRDM12 polypeptide has the activity of positive regulation of H3-K9 dimethylation. In some embodiments, activity of the PRDM12 polypeptide recruits the G9a H3-K9 methyltransferase.

In some embodiments, the PRDM12 polypeptide has the activity of a transcription factor. In some embodiments, the activity of the PRDM12 polypeptide regulates the expression of genes. In some embodiments, the PRDM12 polypeptide has the activity of a developmental transcription factor. In some embodiments, the activity of the PRDM12 polypeptide represses the expression of genes. For example, in some embodiments, the activity of the PRDM12 polypeptide represses the expression DBX1 gene that encodes the Homeobox protein DBX1, also known as developing brain homeobox protein 1. In some embodiments, the activity of the PRDM12 polypeptide represses the expression of the NKX6-1 gene that encodes the Homeobox protein Nkx-6.1.

In some embodiments, the activity of the PRDM12 polypeptide regulates the expression of genes that are markers of cells involved in the neural processes of encoding and processing noxious stimuli, also known as nociception. In some embodiments, the activity of the PRDM12 polypeptide regulates the expression of genes that are markers of nociceptive neurons. For example, in some embodiments, the activity of the PRDM12 polypeptide regulates the expression of the Ntrk1 gene. In some embodiments, the activity of the PRDM12 polypeptide regulates the expression of the Nhlh1 gene. In some embodiments, the activity of the PRDM12 polypeptide regulates the expression of the Brn3a gene. In some embodiments, the activity of the PRDM12 polypeptide regulates the expression of the Neurod1 gene.

In some embodiments, the PRDM12 polypeptide has neurogenesis activity. In some embodiments, the PRDM12 polypeptide is active in the development of the progenitors of sensory neurons. In some embodiments, the PRDM12 polypeptide is active in the development of the neural crest cell progenitors of sensory neurons. In some embodiments, the PRDM12 polypeptide is active in the development of sensory neurons. In some embodiments, the PRDM12 polypeptide is active in the development of the neural crest progenitors of nociceptive sensory neurons. In some embodiments, the PRDM12 polypeptide is active in the development of nociceptive sensory neurons. In some embodiments, the PRDM12 polypeptide is active in the development of nerve-endings. In some embodiments, the PRDM12 polypeptide is active in the development of the nerve-endings of nociceptive sensory neurons. In some embodiments, the PRDM12 polypeptide is active in the development of the neuron projections. In some embodiments, the PRDM12 polypeptide is active in the development of sensory neuron projections. In some embodiments, the PRDM12 polypeptide is active in the development of nociceptive sensory neuron projections.

In some embodiments, activity of the PRDM12 polypeptide is involved in the sensory perception of pain, also known as nociception.

The polypeptide encoded by the exogenous gene expressed in the recombinant cell described herein may be an active fragment of either a polypeptide comprising an amino acid sequence having at least 60% sequence identity to the amino acid sequence of SEQ ID NO. 1, or an active fragment of the PRDM12 polypeptide. In some instances, the active fragment (e.g., an active fragment of an amino acid sequence that has at least 60% sequence identity to the amino acid sequence of SEQ ID NO. 1 or of the PRDM12 polypeptide) is a fragment that has at least one biological activity of either polypeptide, for example the active fragment that is or acts as a transcription factor or of any other biological. In some instances, the PRMD12 polypeptide fragment has a length of at least about 30 amino acid residues.

In one embodiment the fragment has at least 50% of the activity of the full-length protein, such as 60, 65, 70, 75, 80, 85, 90, 95 or 100% of the activity of the full-length protein.

In some embodiments, the recombinant cell provided herein that expresses an exogenous PRDM12 polypeptide or an active fragment thereof is a mammalian cell. In some embodiments, the recombinant cell provided herein that expresses an exogenous PRDM12 polypeptide or an active fragment thereof is a human cell.

In some embodiments, the recombinant cell provided herein is a stem cell (SC). In some specific embodiments, the recombinant cell is an embryonic stem cell (ESC). In more specific embodiments, the recombinant cell is a human embryonic stem cell (hESC). In some embodiments, the recombinant cell provided herein is a pluripotent stem cell (PSC). In specific embodiments, the recombinant cell provided herein is an induced pluripotent stem cell (iPSC). In still more specific embodiments, the recombinant cell provided herein is a human induced pluripotent stem cell (hiPSC). In still more specific embodiments, the recombinant cell provided herein is an XCL-1 hiPSC.

In some embodiments, the recombinant cell is derived from a stem cell (SC). In some embodiments, the recombinant cell is derived from a hiPSC. In some embodiments, the recombinant cell is derived from an XCL-1 hiPSC. In some embodiments, the recombinant cell is a nociceptor derived from an XCL-1 hiPSC. In some embodiments, the recombinant cell is a progenitor cell derived from an SC. In some embodiments, the recombinant cell is an NPC derived from an XCL-1 hiPSC. In some embodiments, the recombinant cell is a multipotent cell derived from a pluripotent stem cell, e.g., an NPC derived from an iPSC.

In some embodiments, the recombinant cell provided herein that expresses an exogenous PRDM12 polypeptide or an active fragment thereof is a neural cell. In some embodiments, the recombinant cell is a neural progenitor cell (NPC). In some embodiments, the recombinant cell is a glial cell. In some specific embodiments, the recombinant cell is a neuron. In some more specific embodiments, the recombinant cell is a sensory neuron. In some even more specific embodiments, the recombinant cell is an afferent sensory neuron. In some specific embodiments, the recombinant cell is a nociceptive lineage sensory neuron. In some specific embodiments, the recombinant cell is a nociceptive sensory neuron. In some specific embodiments, the recombinant cell is a nociceptor-like sensory neuron. In some specific embodiments, the recombinant cell is a nociceptor.

In some embodiments, the recombinant cell is a neural cell (e.g., a neural progenitor cell (NPC) or a neuron). In some instances, the neural cell is a cell that developed in an organism (e.g., a glial cell in the spinal cord or a living rat or a glial cell in a postmortem neural cell culture). In some instances, the neural cell is a cell that was propagated in vitro (e.g., a neural cell derived from a cell culture). In some embodiments, the neural cell that was propagated in vitro has properties of a neuron that developed in an organism.

In some instances, the recombinant cell is a neuron that developed in an organism (e.g., a neuron derived from an organism, such as a neuron in the brain of a living human or a neuron in a postmortem neural cell culture). In some instances, the neuron is a cell that was propagated in vitro (e.g., a neuron derived from a cell culture). In some embodiments, the neuron that was propagated in vitro has properties of a neuron that developed in an organism.

In some instances, the recombinant cell is a sensory neuron is that developed in an organism (e.g., a sensory neuron derived from an organism, such as a sensory neuron in the retina of a living mouse or a sensory neuron in a postmortem neural cell culture). In some instances, the sensory neuron is a cell that was propagated in vitro (e.g., a sensory neuron derived from a cell culture). In some embodiments, the sensory neuron that was propagated in vitro has properties of a sensory neuron that developed in an organism.

In some instances, the recombinant cell is a nociceptor (e.g., a nociceptive sensory neuron). In some instances, the nociceptor is a cell that developed in an organism (e.g., a nociceptor derived from an organism, a nociceptor that has a soma in the dorsal root ganglion (DRG) of a living rat or a DRG nociceptor in a postmortem neural cell culture). In some instances, the nociceptor is a cell that was propagated in vitro (e.g., a nociceptor derived from a cell culture). In some embodiments, the nociceptor that was propagated in vitro has properties of a nociceptor that developed in an organism.

Provided herein is recombinant cell that is capable of proliferating in vitro. In some embodiments, the recombinant cell provided herein is capable of proliferation in cell culture. In some embodiments, the recombinant cell provided herein can propagate repeatedly in a continuous cell culture. In some embodiments, the recombinant cell provided herein can propagate indefinitely or for decades in a continuous cell culture. In some embodiments, the recombinant cell provided herein can propagate for months to years in a continuous cell culture. In some embodiments, the recombinant cell provided herein can propagate for one or more weeks in a continuous cell culture.

In some aspects, provided herein are methods of recombinantly producing a genetically modified cell. In some embodiments, the method comprises providing an isolated cell, and modifying the isolated cell with one or more exogenous genes, wherein the modified cell comprises one or more phenotypic characteristics specific to a sensory neuron. In some embodiments, the one or more exogenous genes is a positive regulatory domain containing (PRDM) gene. In some embodiments, the PRDM gene is PRDM12.

In some embodiments, the modification is performed via gene knock-in (e.g. knock-in of an exogenous sequence into the genome of the isolated cell. In some embodiments, the gene knock-in is at a defined locus (e.g., at the AAVS1 locus). In some embodiments, the method comprises knock-in of a nucleic acid encoding an exogenous PRDM12 polypeptide or active fragment thereof. In some embodiments, the knock-in of the nucleic acid encoding an exogenous PRDM12 polypeptide or active fragment thereof is at the AAVS1 locus.

In some embodiments, a gene-editing system is used to introduce the nucleic acid encoding the exogenous PRDM12 polypeptide or active fragment thereof into the recombinant cell. In some specific embodiments, the gene-editing system comprises an engineered nuclease and a DNA donor template. In some specific embodiments, the gene-editing system comprises an engineered nuclease that is a zinc finger nuclease (ZFN). In some specific embodiments, the gene-editing system comprises an engineered nuclease that is a transcription activator-like effector nuclease (TALEN). In some specific embodiments, the gene-editing system comprises an engineered nuclease that is an RNA-guided engineered nuclease (RGEN) that includes a Cas protein and a guide RNA.

In some embodiments, a CRISPR/Cas gene-editing systems can be used to insert (“knock-in”) a nucleotide sequence that encodes the exogenous PRDM12 polypeptide or an active fragment thereof into the genome of the recombinant cell or into the genome of a progenitor of the recombinant cell. In some embodiments, the CRISPR/Cas gene-editing system comprises (i) a guide RNA (gRNA) complementary to a target sequence or a DNA encoding the gRNA complementary to a target sequence, (ii) a nucleic acid encoding a Cas nuclease or the Cas nuclease itself, and (iii) a DNA donor template.

In some embodiments, the gRNA is a dual RNA comprising CRISPR RNA (crRNA) and trans-activating CRISPR RNA (tracrRNA). In some embodiments, the gRNA is a single-strand gRNA.

In some embodiments, the gRNA comprises a sequence complementary to an AAVS1 safe harbor site (e.g., intron 1 of the PPP1R12C gene) target sequence.

In some embodiments, the CRISPR/Cas system is type I. In one embodiment, the type 1 CRISPR/Cas system comprises Cas3.

In some embodiments, the CRISPR/Cas system is type II. In one embodiment, the type II CRISPR/Cas system comprises Cas9.

In some embodiments, the CRISPR/Cas system is type V. In one embodiment, the type V CRISPR/Cas system comprises Cpf1.

In some embodiments, the DNA donor template is a DNA donor plasmid. In some embodiments, the DNA donor plasmid comprises left and right homology arms flanking a DNA donor insert. In some embodiments, the DNA donor plasmid comprises left and right AAVS1 safe harbor site homology arms flanking a DNA donor insert. In some embodiments, the DNA donor insert comprises a nucleotide sequence that encodes a polypeptide comprising an amino acid sequence having at least 60% sequence identity to an amino acid sequence of SEQ ID NO. 1. In some specific embodiments, the DNA donor insert comprises a nucleotide sequence that encodes a polypeptide comprising an amino acid sequence having at least 60% sequence identity to an amino acid sequence of SEQ ID NO. 1 under the control of an EF1-α promoter. In some embodiments, the DNA donor insert comprises a nucleotide sequence that encodes the PRDM12 polypeptide or an active fragment thereof. In some embodiments, the DNA donor insert comprises a nucleotide sequence that encodes the PRDM12 polypeptide or an active fragment thereof under the control of an EF1-α promoter.

In some aspects, described herein is a method of identifying a drug candidate for treating pain. In some embodiments, the method for identifying a drug candidate for treating pain comprises providing a recombinant cell comprising an exogenous gene, wherein the recombinant cell is capable of proliferating in vitro; culturing the recombinant cell in vitro; contacting the recombinant cell with the drug candidate; evaluating at least one of (i) an electrophysiological characteristic of the recombinant cell, (ii) a proteomic characteristic of the recombinant cell, (iii) a transcriptomic characteristic of the recombinant cell, (iv) a metabolomic characteristic of the recombinant cell, (v) a cell morphology characteristic of the recombinant cell, (vi) an axonal outgrowth characteristic of the recombinant cell, or (vii) cell viability, thereby providing a first measurement; and determining a drug candidate score for the drug candidate based at least in part on the first measurement. In some embodiments, the recombinant cell is any one of the recombinant cells described herein.

In some embodiments, the method for identifying a drug candidate for treating pain comprises providing a recombinant cell comprising an exogenous gene, the exogenous gene encoding a polypeptide comprising an amino acid sequence having at least 60% sequence identity to an amino acid sequence of SEQ ID NO. 1 or an active fragment thereof, wherein the recombinant cell is capable of proliferating in vitro; culturing the recombinant cell in vitro; contacting the recombinant cell with the drug candidate; evaluating at least one of (i) an electrophysiological characteristic of the recombinant cell, (ii) a proteomic characteristic of the recombinant cell, (iii) a transcriptomic characteristic of the recombinant cell, (iv) a metabolomic characteristic of the recombinant cell, (v) a cell morphology characteristic of the recombinant cell, (vi) an axonal outgrowth characteristic of the recombinant cell, or (vii) cell viability, thereby providing a first measurement; and determining a drug candidate score for the drug candidate based at least in part on the first measurement.

In some embodiments, the method for identifying a drug candidate for treating pain comprises providing a recombinant cell comprising an exogenous gene, the exogenous gene encoding a PR domain zinc finger protein 12 (PRDM12) polypeptide, wherein the recombinant cell is capable of proliferating in vitro; culturing the recombinant cell in vitro; contacting the recombinant cell with the drug candidate; evaluating at least one of (i) an electrophysiological characteristic of the recombinant cell, (ii) a proteomic characteristic of the recombinant cell, (iii) a transcriptomic characteristic of the recombinant cell, (iv) a metabolomic characteristic of the recombinant cell, (v) a cell morphology characteristic of the recombinant cell, (vi) an axonal outgrowth characteristic of the recombinant cell, or (vii) cell viability, thereby providing a first measurement; and determining a drug candidate score for the drug candidate based at least in part on the first measurement.

In some instances, pain is nociception. In some instances, nociception refers to the neural encoding and processing of noxious stimuli, such as stimuli produced by impending or actual tissue damage. In some instances, pain is the subjective experience of actual or impending harm. In some instances, pain is both nociception and the subjective experience of actual or impending harm. Though nociceptive stimulation can lead to the subjective experience of actual or impending harm, in some instances, one can exist without the other.

In some embodiments, the method of identifying a drug candidate for treating pain provided herein identifies a drug candidate for treating chronic pain. In some embodiments, the method of identifying a drug candidate for treating pain identifies a drug candidate for treating acute pain. In some embodiments, the method of identifying a drug candidate for treating pain identifies a drug candidate for treating neuropathic pain (e.g., phantom pain). In some embodiments, the method of identifying a drug candidate for treating pain identifies a drug candidate for treating nociceptive pain. In some embodiments, the method of identifying a drug candidate for treating pain identifies a drug candidate for treating radicular pain, such as sciatica. In some embodiments, the method of identifying a drug candidate for treating pain identifies a drug candidate for treating nociplastic pain. In some embodiments, the method of identifying a drug candidate for treating pain identifies a drug candidate for treating psychogenic pain.

In some embodiments, the method of identifying a drug candidate for treating pain identifies a drug candidate for treating visceral pain. In some embodiments, the method of identifying a drug candidate for treating pain identifies a drug candidate for treating somatic pain. In some embodiments, the method of identifying a drug candidate for treating pain identifies a drug candidate for treating pain associated with ischemia (e.g., pain felt or perceived by an individual having, e.g., diagnosed with, ischemia). In some embodiments, the method of identifying a drug candidate for treating pain identifies a drug candidate for treating pain caused associated with inflammation (e.g., pain felt or perceived by an individual having, e.g., diagnosed with, inflammation).

In some embodiments, the method of identifying a drug candidate for treating pain provided herein identifies a drug candidate for treating pain associated with a medical procedure, e.g., pain experienced during a medical procedure. In some embodiments, the method of identifying a drug candidate for treating pain identifies a drug candidate for treating surgical pain, e.g., pain associated with or experienced during a surgery. In some embodiments, the method of identifying a drug candidate for treating pain identifies a drug candidate for treating post-surgical pain. In some specific embodiments, the method of identifying a drug candidate for treating pain identifies a drug candidate for treating post-amputation pain.

In some embodiments, the method of identifying a drug candidate for treating pain identifies a drug candidate for treating pain associated with injury (e.g., pain felt or perceived by an individual having an injury, such as a broken bone, a dislocated joint, or a torn ligament). In some embodiments, the method of identifying a drug candidate for treating pain identifies a drug candidate for treating pain associated with tissue damage (e.g., pain felt or perceived by an individual having tissue damage). In some embodiments, the method of identifying a drug candidate for treating pain identifies a drug candidate for treating pain associated with a wound (e.g., pain felt or perceived by an individual having a wound, such as a puncture wound, a laceration wound, or an abrasion wound). In some embodiments, the method of identifying a drug candidate for treating pain identifies a drug candidate for treating pain associated with exposure to heat (e.g., pain felt or perceived by an individual who was exposed to heat, such as the pain associated with a burn). In some embodiments, the method of identifying a drug candidate for treating pain identifies a drug candidate for treating pain associated with exposure to cold (e.g., pain felt or perceived by an individual who was exposed to cold, such as the pain associated with frostbite). In some embodiments, the method of identifying a drug candidate for treating pain identifies a drug candidate for treating pain associated with exposure to an acidic substance (e.g., pain felt or perceived by an individual who was exposed to an acid, such as the pain associated with a chemical burn). In some embodiments, the method of identifying a drug candidate for treating pain identifies a drug candidate for treating pain associated with exposure to an alkaline substance (e.g., pain felt or perceived by an individual who was exposed to a base, such as the pain associated with a chemical burn).

In some embodiments, the method of identifying a drug candidate for treating pain identifies a drug candidate for treating pain associated with cancer (e.g., pain felt or perceived by an individual having, e.g., diagnosed with, cancer, or pain that is perceived concurrent with or when suffering from cancer). In some embodiments, the method of identifying a drug candidate for treating pain identifies a drug candidate for treating pain associated with tension headache (e.g., pain felt or perceived by an individual having a tension headache, or pain that is perceived concurrent with or when suffering from tension headache). In some embodiments, the method of identifying a drug candidate for treating pain identifies a drug candidate for treating pain associated with migraine headache (e.g., pain felt or perceived by an individual having a migraine headache, or pain that is perceived concurrent with or when suffering from migraine headache). In some embodiments, the method of identifying a drug candidate for treating pain identifies a drug candidate for treating pain associated with fibromyalgia (e.g., pain felt or perceived by an individual having, e.g., diagnosed with, fibromyalgia, or pain that is perceived concurrent with or when suffering from fibromyalgia). In some embodiments, the method of identifying a drug candidate for treating pain identifies a drug candidate for treating pain associated with arthritis (e.g., pain felt or perceived by an individual having, e.g., diagnosed with, arthritis, or pain that is perceived concurrent with or when suffering from arthritis). In some embodiments, the method of identifying a drug candidate for treating pain identifies a drug candidate for treating pain associated with diabetes (e.g., pain felt or perceived by an individual having, e.g., diagnosed with, diabetes, or pain that is perceived concurrent with or when suffering from diabetes). In some embodiments, the method of identifying a drug candidate for treating pain identifies a drug candidate for treating pain associated with ischemic stroke (e.g., pain felt or perceived by an individual having an ischemic stroke, or pain that is perceived concurrent with or when suffering from an ischemic stroke). In some embodiments, the method of identifying a drug candidate for treating pain identifies a drug candidate for treating pain associated with heart attack (e.g., pain felt or perceived by an individual having a heart attack, or pain that is perceived concurrent with or when suffering from a heart attack).

Described herein is a method of identifying a drug candidate for treating pain, the method comprising providing either (i) a recombinant cell comprising an exogenous gene, the exogenous gene encoding a polypeptide comprising an amino acid sequence having at least 60% sequence identity to an amino acid sequence of SEQ ID NO. 1 or an active fragment thereof, wherein the recombinant cell is capable of proliferating in vitro, or (ii) providing a recombinant cell comprising an exogenous gene, the exogenous gene encoding a PR domain zinc finger protein 12 (PRDM12) polypeptide, wherein the recombinant cell is capable of proliferating in vitro. In some embodiments, method further comprises: culturing the recombinant cell in vitro; contacting the recombinant cell with the drug candidate; evaluating (i) an electrophysiological characteristic of the recombinant cell, (ii) a proteomic characteristic of the recombinant cell, or (iii) both, thereby providing a first measurement; and determining a drug candidate score for the drug candidate based at least in part on the first measurement.

In some embodiments, the recombinant cell is cultured in a well plate. In some embodiments, the recombinant cell is cultured in a culture flask. In some embodiments, the recombinant cell is cultured in a culture dish. In some embodiments, the recombinant cell is cultured in a medium that comprises gelatin. In some embodiments, the recombinant cell is cultured in a medium that comprises feeder medium. In some embodiments, the recombinant cell is cultured in human embryonic stem cell medium. In some embodiments, the recombinant cell is cultured in a medium that comprises Dulbecco's Modified Eagle Medium/Nutrient Mixture F-12 (DMEM-F12). In some embodiments, the recombinant cell is cultured in a medium that comprises knock-out serum replacer (KOSR). In some embodiments, the recombinant cell is cultured in a medium that comprises laminin. In some embodiments, the recombinant cell is cultured in a medium that comprises an antibiotic. In some specific embodiments, the recombinant cell is cultured in a medium that comprises penicillin. In some specific embodiments, the recombinant cell is cultured in a medium that comprises streptomycin. In some embodiments, the recombinant cell is cultured in a medium that comprises phosphate buffered saline (PBS). In some embodiments, the recombinant cell is cultured at a temperature between about 15° C. to 40° C. In some specific embodiments, the recombinant cell is cultured at a temperature between about 30° C. to 40° C. In some still more specific embodiments, the recombinant cell is cultured at a temperature between about 35° C. to 40° C. In some still more specific embodiments, the recombinant cell is cultured at a temperature of about 37° C. In some specific embodiments, the recombinant cell is cultured at a temperature between about 15° C. to 30° C. In some still more specific embodiments, the recombinant cell is cultured at a temperature between about 17° C. to 25° C. In some still more specific embodiments, the recombinant cell is cultured at a temperature of about 20° C. In some embodiments, the recombinant cell is cultured in air that contains between about 3% CO2 to 7% CO2. In some embodiments, the recombinant cell is cultured in air that contains about 5% CO2.

In some embodiments, the recombinant cell is contacted with the drug candidate by providing the drug candidate in the culture medium. In some embodiments, the recombinant cell is contacted with the drug candidate by perfusing the drug candidate onto the recombinant cell. In some embodiments, the recombinant cell is contacted with the drug candidate by incubating the recombinant cell with the drug candidate. In some embodiments, the recombinant cell is contacted with the drug candidate by introducing the drug candidate into cytoplasm of the recombinant cell. In some embodiments, the recombinant cell is contacted with the drug candidate for the entire life of the cell. In some embodiments, the recombinant cell is contacted with the drug candidate for a duration of weeks to months. In some embodiments, the recombinant cell is contacted with the drug candidate for a duration of a day to a week. In some embodiments, the recombinant cell is contacted with the drug candidate for a duration of about 1 to 24 hours. In some embodiments, the recombinant cell is contacted with the drug candidate for a duration of about 1 to 60 minutes. In some embodiments, the recombinant cell is contacted with the drug candidate for a duration of about 1 to 60 seconds. In some embodiments, the recombinant cell is contacted with the drug candidate for less than 1 second.

In some embodiments, the method comprises evaluating at least one of (i) an electrophysiological characteristic of the recombinant cell, (ii) a proteomic characteristic of the recombinant cell, (iii) a transcriptomic characteristic of the recombinant cell, (iv) a metabolomic characteristic of the recombinant cell, (v) a cell morphology characteristic of the recombinant cell, (vi) an axonal outgrowth characteristic of the recombinant cell, or (vii) cell viability, thereby providing a first measurement.

In some embodiments, the method comprises evaluating an electrophysiological characteristic of the cell. In some embodiments, the electrophysiological characteristic is the current through ion channels (e.g., while holding the membrane voltage at a defined level). In some embodiments, the electrophysiological characteristic is ion-channel conductance (e.g., while holding the membrane voltage at a defined level). In some embodiments, the electrophysiological characteristic is membrane conductance (e.g., while holding the membrane voltage at a defined level). In some embodiments, the electrophysiological characteristic is change in membrane conductance (e.g., while holding the membrane voltage at a defined level). In some embodiments, the electrophysiological characteristic is membrane potential. In some embodiments, the electrophysiological characteristic is change in membrane potential. In some embodiments, the electrophysiological characteristic is rate of action potentials. In some embodiments, the electrophysiological characteristic is change in rate of action potentials. In some embodiments, the electrophysiological characteristic is timing of action potentials. In some embodiments, the electrophysiological characteristic is change in timing of action potentials. In some embodiments the change in time of action potentials is a change in the action potential firing rate. In some embodiments the change in time of action potentials is a change in the action potential rising phase. In some embodiments the change in time of action potentials is a change in action potential decay. In some embodiments the change in time of action potentials is a change in the action potential refractory period. In some embodiments, the electrophysiological characteristic is rate of postsynaptic potentials, change in rate of postsynaptic potentials. In some embodiments, the electrophysiological characteristic is timing of postsynaptic potentials. In some embodiments, the electrophysiological characteristic is change in timing of postsynaptic potentials. In some embodiments, the electrophysiological characteristic is a property of an action potential (AP). In some specific embodiments, the electrophysiological characteristic is the AP threshold. In some specific embodiments, the electrophysiological characteristic is the AP rise time. In some specific embodiments, the electrophysiological characteristic is the AP decay time. In some specific embodiments, the electrophysiological characteristic is the AP amplitude.

In some embodiments, a proteomic characteristic of the cell is evaluated. In some embodiments, the evaluated proteomic characteristic of the cell is protein expression. In some embodiments, the evaluated proteomic characteristic of the cell is change in protein expression. In some embodiments, the evaluated proteomic characteristic of the cell is protein expression levels. In some embodiments, the evaluated proteomic characteristic of the cell is change in protein expression levels. In some embodiments, the evaluated proteomic characteristic of the cell is posttranslational modification of proteins. In some embodiments, the evaluated proteomic characteristic of the cell is change in posttranslational modification of proteins. In some embodiments, the evaluated proteomic characteristic is interactions between components of a cell, such as interactions between proteins. In some embodiments, the evaluated proteomic characteristic of the cell is ligand-receptor interactions. In some embodiments, the evaluated proteomic characteristic of the cell is change in ligand-receptor interactions.

In some embodiments, the method comprises evaluating a transcriptomic characteristic of the recombinant cell. In some embodiments, the transcriptomic data is obtained from RNA sequencing, e.g., by RNA-seq. In some embodiments, the transcriptomic data is obtained by microarray.

In some embodiments, the method comprises evaluating a metabolomic characteristic of the recombinant cell. In some embodiments, evaluating a metabolomic characteristic comprises detection, identification and/or quantification of one or more metabolites, e.g., using mass spectrometry and/or nuclear magnetic resonance.

In some embodiments, the method comprises evaluating a cell morphology characteristic of the recombinant cell.

In some embodiments, the method comprises evaluating an axonal outgrowth characteristic of the recombinant cell

In some embodiments, the method comprises evaluating a cell viability characteristic.

In some embodiments, the characteristic of the recombinant cell is evaluated while the recombinant cell is in contact with the drug candidate. In some embodiments, the characteristic of the recombinant cell is evaluated after the recombinant cell is contacted with the drug candidate. In some embodiments, the characteristic of the recombinant cell is evaluated weeks to months after the recombinant cell is contacted with the drug candidate. In some embodiments, the characteristic of the recombinant cell is evaluated a day to a week after the recombinant cell is contacted with the drug candidate. In some embodiments, the characteristic of the recombinant cell is evaluated about 1 to 24 hours after the recombinant cell is contacted with the drug candidate. In some embodiments, the characteristic of the recombinant cell is evaluated about 1 to 60 minutes after the recombinant cell is contacted with the drug candidate. In some embodiments, the characteristic of the recombinant cell is evaluated about 1 to 60 seconds after the recombinant cell is contacted with the drug candidate. In some embodiments, the characteristic of the recombinant cell is evaluated less than a second after the recombinant cell is contacted with the drug candidate.

In some embodiments, the drug candidate score comprises, at least in part, evaluating the first measurement and a second measurement. In some specific embodiments, the second measurement is a standard score for the recombinant cell. In some embodiments, the standard score is a score obtained from a database. In some embodiments, the standard score is obtained from the Molecular Signatures Database (MSigDB). In some embodiments, the standard score is obtained from the Global Proteome Machine Database (GPMDB). In some embodiments, the standard score is obtained from the PeptideAtlas. In some embodiments, the standard score is obtained from the PRIDE database. In some embodiments, the standard score is obtained from the ProteomicsDB. In some embodiments, the standard score is obtained from the Mass Spectrometry Interactive Virtual Environment (MassIVE) database. In some embodiments, the standard score is obtained from the Chorus database. In some embodiments, the standard score is obtained from the MaxQB database. In some embodiments, the standard score is obtained from the PeptideAtlas. In some embodiments, the standard score is obtained from the SRM Experiment Library (PASSEL). In some embodiments, the standard score is obtained from the Model Organism Protein Expression Database (MOPED). In some embodiments, the standard score is obtained from the Human Proteinpedia. In some embodiments, the standard score is obtained from the KEGG PATHWAY database. In some embodiments, the standard score is obtained from an RNA sequencing database.

In some specific embodiments, the second measurement is obtained (at least in part) by evaluating the characteristic before contacting the recombinant cell with the drug candidate. In some embodiments, the second measurement is obtained by evaluating the characteristic of the recombinant cell weeks to months before the recombinant cell is contacted with the drug candidate. In some embodiments, the second measurement is obtained by evaluating the characteristic of the recombinant cell a day to a week before the recombinant cell is contacted with the drug candidate. In some embodiments, the second measurement is obtained by evaluating the characteristic of the recombinant cell about 1 to 24 hours before the recombinant cell is contacted with the drug candidate. In some embodiments, the second measurement is obtained by evaluating the characteristic of the recombinant cell about 1 to 60 minutes before the recombinant cell is contacted with the drug candidate. In some embodiments, the second measurement is obtained by evaluating the characteristic of the recombinant cell about 1 to 60 seconds before the recombinant cell is contacted with the drug candidate. In some embodiments, the second measurement is obtained by evaluating the characteristic of the recombinant cell less than a second before the recombinant cell is contacted with the drug candidate. In some specific embodiments, the second measurement is obtained (at least in part) by evaluating the characteristic simultaneously with contacting the recombinant cell with the drug candidate.

Any aspect or embodiment described herein can be combined with any other aspect or embodiment as disclosed herein.

### EXAMPLES

The following examples are provided to further illustrate embodiments of the present disclosure, but are not intended to limit the scope of the disclosure; it will be understood by their exemplary nature that other procedures, methodologies, or techniques known to those skilled in the art may alternatively be used.

### Example 1: Generation of Clones with Targeted Integration of the PRDM12 Nucleic Acid Coding Sequence in Induced Pluripotent Stem Cells (iPSCs)

XCL-1 induced pluripotent stem cells (iPSCs) were nucleofected with (i) a plasmid containing a CRISPR ribonuclease and an AAVS1 locus-specific single gRNA, and (ii) a PRMD12 DNA donor template (p-EF1α-PRMD12). The PRMD12 DNA donor template contained a codon-optimized PRDM12 gene under the control of the EF1α promoter modified with flanking left and right AAVS1 homology arms. Cell colonies were recovered, and genomic DNA (gDNA) was harvested at 3-5 days post transfection to check for ribonuclease activity and to confirm genome integration of the PRMD12 donor. Cell colonies were cryopreserved.

### Example 2: Single Cell Cloning from Pools of Cells Transfected with Nucleic Acid Sequence Coding for PRDM12

Single cell clones taken from the cryopreserved cell colonies transfected with the PRDM12 nucleic acid sequence (Example 1) were plated out. Clones were allowed to grow for approximately 12 days. DNA was collected from consolidated clones and screened to check for retention of genomic integration of the PRDM12 donor plasmid. All clones with confirmed integration of the PRDM12 donor plasmid were expanded and cryopreserved.

### Example 3: Differentiation and Culture of Recombinant Nociceptors Derived from XCL-1 hiPSCs Expressing Exogenous PRDM12 Polypeptide

Recombinant XCL-1 human iPSCs expressing exogenous PRDM12 polypeptide are differentiated into recombinant nociceptors and cultured as described, in part, in Gunhanlar, et al., 2018.

XCL-1 human iPSC genetically modified to have homozygous insertion of the PRDM12 gene in the AAVS1 safe harbor are dissociated from mouse embryonic fibroblasts with collagenase (100 U ml-1, Thermo Fisher Scientific, Waltham, MA, USA) for 7 min at 37° C./5% CO2. Embryoid bodies (EBs) are generated by transferring dissociated iPSCs to non-adherent plates in human embryonic stem cell medium (Dulbecco's Modified Eagle Medium/Nutrient Mixture F-12 (DMEM)/F12 (Thermo Fisher Scientific), 20% knockout serum (Thermo Fisher Scientific), 1% minimum essential medium/non-essential amino acid (Sigma-Aldrich, St Louis, MO, USA), 7 nl ml−1 β-mercaptoethanol (Sigma-Aldrich), 1% L-glutamine (Thermo Fisher Scientific) and 1% penicillin/streptomycin (Thermo Fisher Scientific)) on a shaker in an incubator at 37° C./5% CO2. EBs are grown for 2 days in human embryonic stem cell medium, are changed into neural induction medium (DMEM/F12, 1% N2 supplement (Thermo Fisher Scientific), 2 μg ml−1 heparin (Sigma-Aldrich) and 1% penicillin/streptomycin) on day 2 (d2) and are cultured for another 4 days in suspension (d3-d6). For generation of neural precursor cells (NPCs), EBs are slightly dissociated at d7 by trituration and are plated onto laminin-coated 10 cm dishes (20 μg ml−1 laminin (Sigma-Aldrich) in DMEM for 30 min at 37° C.), initially using neural induction medium (d7-14), and then from d15 in neural progenitor cell (NPC) medium (DMEM/F12, 1% N2 supplement, 2% B27-RA supplement (Thermo Fisher Scientific), 1 μg ml−1 laminin, 20 ng ml−1 basic fibroblast growth factor (Merck-Millipore, Darmstadt, Germany) and 1% penicillin/streptomycin). On d15, cells are considered pre-NPCs (passage 1) and are able to be passaged (1:4) and cryopreserved when confluent. From passage 5, cells are considered NPCs and used for neural differentiation.

NPCs (passages 5-11) are plated on sterile coverslips in 6- or 12-well plates and coated with poly-L-ornithine (Sigma-Aldrich) for 1 h at room temperature. Coated coverslips are washed 3 times with sterile water and dried for 30 min. Subsequently, a 100 μl drop of laminin solution (50 μg ml−1 in water) is placed in the middle of each coverslip, incubated for 15-30 min at 37° C./5% CO2 and then is replaced with a 100 μl drop of DMEM until plating of NPCs. Immediately before plating, NPCs are washed with Dulbecco's phosphate-buffered saline and are dissociated with collagenase (100 U ml−1). One fully confluent 10 cm dish of NPCs is divided over a 12-well plate. A 100 μl drop of NPC cell suspension is placed on the laminin-coated spot for 1 h to allow for attachment of NPCs on coverslips in neural differentiation medium (Neurobasal medium, 1% N2 supplement, 2% B27-RA supplement, 1% minimum essential medium/non-essential amino acid, 20 ng ml−1 brain-derived neurotrophic factor (ProSpec Bio, Rehovot, Israel), 20 ng ml−1 glial cell-derived neurotrophic factor (ProSpec Bio), 1 μM dibutyryl cyclic adenosine monophosphate (Sigma-Aldrich), 200 μM ascorbic acid (Sigma-Aldrich), 2 μg ml−1 laminin and 1% penicillin/streptomycin). After 1 h, 900 μl of neural differentiation medium is added to each well. Cells are refreshed with medium 3 times per week. During weeks 1-4, medium is fully refreshed. After 4 weeks of neural differentiation, only half of the volume of medium per well is refreshed. In vitro evaluation of drug candidate using electrophysiology and proteomics ire performed between 8 and 10 weeks after plating of NPCs.

### Example 4: In Vitro Evaluation of Recombinant Cell Characteristics Using Electrophysiological Methodologies

Electrophysiological characteristics of recombinant hiPSC-derived nociceptors expressing exogenous PRDM12 polypeptide are evaluated as described, in part, in Gunhanlar, et al., 2018.

Before initiating whole-cell recordings, cell culture medium is gradually replaced with oxygenated artificial cerebral spinal fluid (ACSF) in order to minimize the impact of the relative difference in osmolarity (culture medium, 220 mOsm 1-1; ACSF, 305 mOsm 1-1). Into the 1 ml volume of culture medium per well, 300 μl of oxygenated ACSF is added for 5 min, after which 300 μl is removed. This replacement procedure was repeated 5 times at room temperature. Slides are placed immediately thereafter into the recording chamber with continuous perfusion of oxygenated ACSF.

Culture slides are collected from 6- or 12-well culture plates. Whole-cell patch-clamp recordings of recombinant nociceptors are performed at 8-10 weeks following the initiation of NPC differentiation. Recording micropipettes (tip resistance 3-6 MΩ) are filled with internal solution composed of (in mM): 130 K-gluconate, 0.1 EGTA, 1 MgCl2, 2 MgATP, 0.3 NaGTP, 10 HEPES, 5 NaCl, 11 KCl and 5 Na2-phosphocreatine (pH 7.4). Recordings are made at room temperature using a MultiClamp 700B amplifier (Molecular Devices, Sunnyvale, CA, USA). Signals are sampled and filtered at 10 and 3 kHz, respectively. The whole-cell capacitance is compensated and series resistance is monitored throughout the experiment in order to confirm the integrity of the patch seal and the stability of the recording. Voltage is corrected for liquid junction potential (−14 mV). The bath is continuously perfused with oxygenated ACSF composed of (in mM): 110 NaCl, 2.5 KCl, 2 CaCl2, 10 glucose and 1 NaH2PO4, 25 NaHCO₃, 0.2 ascorbic acid and 2 MgCl2 (pH 7.4). For voltage-clamp recordings, cells are clamped at −80 mV. Spontaneous postsynaptic currents are recorded for 3 min. Fast sodium and potassium currents are evoked by voltage steps ranging from −80 to +50 m V in 10 mV increments. Capacitance is derived from the Clampex 10.2 (Molecular Devices) membrane-test function. For current-clamp recordings, voltage responses are evoked from a holding potential of −75 m V using 500 ms steps ranging from −20 to +150 pA in 10 pA intervals delivered at 0.5 Hz. Single action potential (AP) properties are calculated from the first evoked action potential (AP) in response to a depolarizing step. Repetitively firing nociceptors are defined as those capable of firing at least 3 mature APs without significant accommodation in response to a depolarizing current step.

A perfusion system permits control of the extracellular solution, including the addition of drug candidates to the extracellular solution. Spontaneous AP activity is measured for 3 min using the minimum hyperpolarizing holding current in which spiking is evident (0-10 pA), from an initial holding potential of −80 mV. AP threshold is calculated as the point at which the second derivative of the AP waveform exceeded baseline. AP rise and decay times are calculated between 10% and 90% of the AP amplitude. Data analysis is performed by Clampfit 10.2 (Molecular Devices). Spontaneous postsynaptic currents are analyzed by MiniAnalysis software (Synaptosoft, Fort Lee, NJ, USA).

The in vitro evaluation of a recombinant cell characteristic using electrophysiology constitutes, at least in part, a measurement.

### Example 5: In Vitro Evaluation of Recombinant Cell Characteristics Using Proteomic Methodologies

Proteomic characteristics of recombinant hiPSC-derived nociceptors expressing exogenous PRDM12 polypeptide are evaluate as described, in part, in Wangzhou, et al., 2021.

Single-cell RNA sequencing (scRNA-seq) data of recombinant hiPSC-derived nociceptors expressing exogenous PRDM12 polypeptide is generated, for example, using Smart-seq2 sequencing of specific cells using a fluorescence-activated cell sorting (FACS) method or a microfluidic emulsion method.

Bulk RNA-seq data of recombinant hiPSC-derived nociceptors expressing exogenous PRDM12 polypeptide is generated, for example, by isolating and suspending recombinant nociceptors, and then sequencing recombinant nociceptors using bulk RNA-seq. Normalized read counts per genes are analyzed and a clustering-based cell-type label for each cell is used.

scRNA-seq data and bulk RNA-seq data are used to generate transcriptome profiles of hiPSC-derived nociceptors expressing exogenous PRDM12 polypeptide. Expression values and metadata are used in further analysis.

The in vitro evaluation of a recombinant cell characteristic using proteomics constitutes, at least in part, a measurement.

### Example 6: Determining a Drug Candidate Score for the Drug Candidate Using a Single Measurement

A recombinant hiPSC-derived nociceptor expressing exogenous PRDM12 is cultured in vitro as described in Example 3. The recombinant nociceptor is contacted with the therapeutic agent (e.g., the therapeutic agent is added to the culture medium or perfused onto the cell). An electrophysiological characteristic (as described in Example 4), a proteomic characteristic (as described in Example 5), or both of the recombinant hiPSC-derived nociceptor expressing exogenous PRDM12 polypeptide is evaluated (e.g., while the recombinant nociceptor is in contact with the therapeutic candidate), thereby providing a measurement. A drug candidate score for the drug candidate is determined based, at least in part, on the measurement.

### Example 7: Determining a Drug Candidate Score for the Drug Candidate Using a First and a Second Measurement

A recombinant hiPSC-derived nociceptor expressing exogenous PRDM12 is cultured in vitro as described in Example 3. An electrophysiological characteristic (as described in Example 4), a proteomic characteristic (as described in Example 5), or both of the recombinant hiPSC-derived nociceptor expressing exogenous PRDM12 polypeptide is evaluated, thereby providing a first measurement. The recombinant nociceptor is contacted with the therapeutic agent (e.g., the therapeutic agent is added to the culture medium or perfused onto the cell). The electrophysiological characteristic (as described in Example 4), a proteomic characteristic (as described in Example 5), or both of the recombinant hiPSC-derived nociceptor expressing exogenous PRDM12 polypeptide is evaluated a second time (e.g., while the recombinant nociceptor is in contact with the therapeutic candidate), thereby providing a second measurement. A drug candidate score for the drug candidate is determined based, at least in part, on evaluating the first measurement and the second measurement.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

TABLE 1

| SEQ ID | Sequence | |
|---|---|---|
| NO. | Name | Sequence |
| 1 | Human positive | MMGSVLPAEALVLKTGLKAPGLALAEVITSDILHSFLYGRWRN |
| | regulatory | VLGEQLFEDKSHHASPKTAFTAEVLAQSFSGEVQKLSSLVLPAE |
| | domain zinc | VIIAQSSIPGEGLGIFSKTWIKAGTEMGPFTGRVIAPEHVDICKNN |
| | finger protein | NLMWEVFNEDGTVRYFIDASQEDHRSWMTYIKCARNEQEQNLE |
| | 12 (PRDM12) | VVQIGTSIFYKAIEMIPPDQELLVWYGNSHNTFLGIPGVPGLEED |
| | | QKKNKHEDFHPADSAAGPAGRMRCVICHRGFNSRSNLRSHMRI |
| | | HTLDKPFVCRFCNRRFSQSSTLRNHVRLHTGERPYKCQVCQSAY |
| | | SQLAGLRAHQKSARHRPPSTALQAHSPALPAPHAHAPALAAAA |
| | | AAAAAAAHHLPAMVL |

1. A recombinant cell comprising an exogenous gene, the exogenous gene encoding a polypeptide comprising an amino acid sequence having at least 60% sequence identity to an amino acid sequence of SEQ ID NO. 1 or an active fragment thereof, wherein the recombinant cell is capable of proliferating in vitro.

2. (canceled)

3. The recombinant cell of claim 1, wherein the recombinant cell forms colonies that propagate repeatedly.

4. The recombinant cell of claim 1, wherein the recombinant cell is a stem cell.

5. The recombinant cell of claim 1, wherein the recombinant cell is a pluripotent stem cell (PSC).

6. The recombinant cell of claim 1, wherein the recombinant cell is an induced pluripotent stem cell (iPSC).

7. The recombinant cell of claim 1, wherein the recombinant cell is a human induced pluripotent stem cell (hiPSC).

8. The recombinant cell of claim 1, wherein the recombinant cell is an XCL-1 hiPSC.

9. The recombinant cell of claim 1, wherein the recombinant cell is a neural cell.

10. The recombinant cell of claim 9, wherein the neural cell is a neural progenitor cell (NPC).

11. The recombinant cell of claim 9, wherein the neural cell is a neuron.

12. The recombinant cell of claim 11, wherein the neuron is a sensory neuron.

13. The recombinant cell of claim 12, wherein the neuron is an afferent sensory neuron.

14. The recombinant cell of claim 12, wherein the neuron is a nociceptive lineage sensory neuron.

15. The recombinant cell of claim 12, wherein the neuron is a nociceptive sensory neuron.

16. The recombinant cell of claim 12, wherein the neuron is a nociceptor-like sensory neuron.

17. The recombinant cell of claim 12, wherein the neuron is a nociceptor.

18. The recombinant cell of claim 1, wherein the polypeptide comprises an amino acid sequence with at least 80% sequence identity to SEQ ID NO: 1.

19.-23. (canceled)

24. The recombinant cell of claim 1, wherein the polypeptide comprises a transcription factor domain.

25-27. (canceled)

28. A method of identifying a drug candidate for treating pain, the method comprising:
a. providing a recombinant cell comprising an exogenous gene, the exogenous gene encoding a polypeptide comprising an amino acid sequence having at least 60% sequence identity to an amino acid sequence of SEQ ID NO. 1 or an active fragment thereof, wherein the recombinant cell is capable of proliferating in vitro;
b. culturing the recombinant cell in vitro;
c. contacting the recombinant cell with the drug candidate;
d. evaluating at least one of (i) an electrophysiological characteristic of the recombinant cell, (ii) a proteomic characteristic of the recombinant cell, (iii) a transcriptomic characteristic of the recombinant cell, (iv) a metabolomic characteristic of the recombinant cell, (v) a cell morphology characteristic of the recombinant cell, (vi) an axonal outgrowth characteristic of the recombinant cell, or (vii) cell viability, thereby providing a first measurement; and
e. determining a drug candidate score for the drug candidate based at least in part on the first measurement.

29. A method of identifying a drug candidate for treating pain, the method comprising:
a. providing a recombinant cell comprising an exogenous gene, the exogenous gene encoding a PR domain zinc finger protein 12 (PRDM12) polypeptide, wherein the recombinant cell is capable of proliferating in vitro;
b. culturing the recombinant cell in vitro;
c. contacting the recombinant cell with the drug candidate;
d. evaluating at least one of (i) an electrophysiological characteristic of the recombinant cell, (ii) a proteomic characteristic of the recombinant cell, (iii) a transcriptomic characteristic of the recombinant cell, (iv) a metabolomic characteristic of the recombinant cell, (v) a cell morphology characteristic of the recombinant cell, (vi) an axonal outgrowth characteristic of the recombinant cell, or (vii) cell viability, thereby providing a first measurement; and
e. determining a drug candidate score for the drug candidate based at least in part on the first measurement.

30-59. (canceled)
